(19) United States
(12) Patent Application Publication
(10) Pub. No.: US 2009/0129975 A1
(43) Pub. Date: May 21, 2009

(54) Apparatus for Sterilizing Components of Packaging Units, Particularly Bottles and/or Caps

(75) Inventors: Luca Colato, Monticelli Terme (IT); Angelo Silvestri, Ricco del Golfo (IT)

Correspondence Address: NIXON & VANDERHYE, PC, 901 NORTH GLEBE ROAD, 11TH FLOOR, ARLINGTON, VA, 22203, US

(73) Assignee: SIDEL S.p.A., Parma (IT)

(21) Appl. No.: 11/921,453

(22) PCT Filed: May 31, 2006
(86) PCT No.: PCT/EP2006/062772

§ 371 Date: Apr. 10, 2008

(30) Foreign Application Priority Data
Jun. 3, 2005 (IT) MO2005A000138

(51) Int. Cl. A61L 2/04; A61L 2/00

(52) U.S. Cl. 422/38; 422/302; 422/291

(57) ABSTRACT

An apparatus for treating objects comprises a plurality of rotating carousels for transporting said objects along a curved path, sterilizing devices arranged on said plurality of carousels along said path and distributed on a carousel of said plurality of carousels and partially on at least a further carousel of said plurality of carousels; an apparatus for treating objects comprises a rotating carousel arrangement for transporting said objects along a curved path, said rotating carousel arrangement comprising at least a carousel for treating objects, and a sterilizing arrangement arranged on said carousel arrangement along said path, and comprising a sterilizing device that operates at ambient pressure; a method for treating objects comprises transporting along a curved path said objects by means of a plurality of rotating carousels comprising sterilizing said objects during said transporting with sterilizing devices arranged on said plurality of carousels, wherein said transporting comprises transferring said objects between a first carousel of said plurality of carousels and at least a further carousel of said plurality of carousels.

This application is a National Phase Application of PCT International Application No. PCT/EP2006/062772 filed May 31, 2006 and claims priority to IT Application No. MO2005A000138 filed Jun. 3, 2005. The entire contents of these applications are incorporated herein by reference.

The invention relates to an apparatus and methods for treating components of packaging units, in particular bottles, and/or caps. These apparatuses and methods are particularly suitable for being used for sterilising bottles filled in aseptic conditions.

In producing containers filled in an aseptic manner, in particular containers intended to be filled with foodstuffs such as milk or fruit juices, or other drinks, it is on the one hand very important to ensure that both the containers and the contents thereof, and the caps are sterile and safe for the health of the consumer, on the other hand it is very important to maintain the organoleptic features of these substances.

For this purpose, it is necessary to sterilise the containers, both internally and externally, before filling them, sterilising the caps with which to close the containers, filling and closing the containers in an aseptic environment in order to avoid contamination thereof after sterilisation.

Various sterilising techniques of containers to be filled are known that use various sterilising agents, depending on the final use of the containers and on the features of the substances contained in the treated containers.

For example, in the case of containers filled with fruit juices with added vitamins that are thermolabile or sensitive to heat variations and to the presence of oxygen it is necessary to treat the filled containers in such a way that the vitamin content in the juices does not decrease and thus avoid heat treatments or treatments sensitive to temperature variations and avoid operating in the presence of oxygen.

In particular, using a hydrogen peroxide solution is known that is injected inside the containers to be sterilised and is left in contact with the internal surface of the containers for a period that is sufficient to bring about the sterilisation reaction. When this time has passed, the containers are treated so as to eliminate the residues of hydrogen peroxide.

From WO02/072158 is known using a hydrogen peroxide solution for sterilising bottles moving on a treatment carousel. The bottles are first inserted in a sterilising chamber that is sealed, subsequently the pressure of the sterilising chamber is varied and then into each bottle a mixture of water steam and hydrogen peroxide vapour are introduced; condensation of the mixture of vapour and steam is awaited, in consequence of which sterilisation occurs. Therefore the condensates are extracted through the effect of the vacuum and the bottles are subjected to a current of sterile air or sterile oxygen that enables the residues of hydrogen peroxide to be removed.

A drawback of the apparatus disclosed in WO02/72158 is that the operations provide for varying the pressure to which the bottles to be sterilised are subjected, first in the sterilising chamber, and subsequently to extract the condensates of the sterilising solution.

These operations entail considerable loss of time and a considerable financial outlay.

The hydrogen peroxide sterilising solution that is normally stored in liquid state, has to be vaporised before being introduced into the bottles to be sterilised to improve the sterilising operation.

For this purpose, in the prior art, is known making a sterilising unit by winding an electric resistor around each conveying conduit that is provided in a sterilising apparatus for conveying the sterilising solution to an injecting nozzle connected to a bottle to be sterilised.

When the sterilising solution has to be introduced into a bottle, a valve of the conveying conduit is opened and a volumetric dosing system dispenses a determined quantity of solution that had previously accumulated in a suitable accumulating chamber to flow into the conveying conduit.

Simultaneously, the resistor is heated in a virtually instant manner so that it can transfer to the solution that is flowing in the conduit the heat required to make it vaporise.

A drawback of an installation of this type is that each resistor has to heat and vaporise the solution within a very short time, by absorbing from the electrical network alone all the necessary power.

The electric power absorbed by each resistance will therefore be equal, except for possible losses, to the installed power, which consequently has to be very high to heat each resistor instantaneously.

This fact places a great limit on the treating apparatuses for industrial products in which simultaneously a considerable number of containers to be sterilised are treated, especially when rotating machines are used, as in such apparatuses high production speeds are required and many sterilising units are installed for vaporising the sterilising solution, each of these units is provided with a respective electrical resistance.

Further, all the resistors provided in the same treatment apparatus have to be connected to a collector that has to be transported with the rotating machine, and that takes up the current from the electrical network and transfers it to the resistors that absorb it in turn.

Further, between the collector and the resistors a current transformer has to be interposed, which is also to be transported with the rotating machine, as the collector absorbs current from the electrical network at a voltage of 380 V, whilst the resistances absorb current at 220V.

During the warm-up phase of such apparatuses all the resistances provided on the same machine (controlled with the PWM method) have to be simultaneously supplied and so the total electric power installed on the apparatus overall can reach very extremely high values. Further, the collectors and transformers provided for these apparatuses have to have great dimensions and are difficult to transport on rotating parts.

During the operative phase of these apparatuses not all the resistances provided are supplied simultaneously but the number of resistances supplied at the same moment is nevertheless such as to require installed electric power values that are so high that the collectors and the transformers have dimensions that are such that they are difficult to transport on rotating parts.

Thus, in providing installations according to the prior art, as previously disclosed, in rotating machines, numerous technical obstacles are encountered, the most obvious of which are:
the need to use, on the rotating parts of the apparatuses, electrical brush collectors of large dimensions with consequent problems of bulk, weight, cost, reliability, maintenance, etc. . . . ;
the need to use, on the rotating parts of the apparatuses, large 380V-220V transformers with consequent problems of bulk, weight, cost, reliability, maintenance, etc. . . .

Such installation is thus difficult to provide on rotating carousels, especially on carousels intended to treat a great number of bottles.

A further drawback of known apparatuses consists of the dosing system of the sterilising solution.

In fact, volumetric dosing systems enable a flow rate of solution to be obtained at the outlet that is not constant over time but variable according to a step function.

This causes oscillations in the temperature reached by the solution and, therefore, in the degree of vaporisation thereof and can negatively influence the efficacy of sterilisation.

Further, to keep the temperature of the solution leaving the heating system constant, it would be necessary to vary the instantaneous power absorbed by the resistances, and this may further increase the peaks of instant power.

An object of the invention is to improve treatment apparatuses of components of packaging units.

A further object is to make treatment apparatuses for treating components of packaging units that are cheap, but also flexible, i.e. which can easily adapt to possible variations in productive conditions without causing rejects.

Still another object is to make treatment apparatuses provided with improved systems of vaporisation of a sterilising solution.

Still another object is to provide treatment apparatuses for treating components of packaging units provided with vaporisation systems of a sterilising solution of moderate cost.

Still another object is to make treatment apparatuses provided with dosing systems of the sterilising solution that enable sterilising solution to be supplied at a flow rate that is constant over time.

In a first aspect of the invention, an apparatus is provided for treating objects comprising a plurality of rotating carousels for transporting said objects along a curved path, sterilising devices arranged on said plurality of carousels along said path, wherein said sterilising devices are distributed on a carousel of said plurality of carousels and partially on at least a further carousel of said plurality of carousels.

In a second aspect of the invention, a method is provided for treating objects comprising transporting along a curved path said objects by means of a plurality of rotating carousels comprising sterilising said objects during said transporting with sterilising devices arranged on said plurality of carousels, wherein said transporting comprises transferring said objects between a first carousel of said plurality of carousels and at least a further carousel of said plurality of carousels.

Owing to these aspects of the invention, it is possible to make versatile and safe treatment apparatus and methods for treating components of packaging units that enable sterile components of packaging units to be obtained.

These apparatuses and methods adapt easily to treating different types of packaging units that are also of dimensions and features that are very different from one another.

In a third aspect of the invention, an apparatus is provided for treating objects comprising a rotating carousel arrangement for transporting said objects along a curved path, said rotating carousel arrangement comprising at least a carousel for treating objects, and a sterilising arrangement arranged on said carousel arrangement along said path, wherein said sterilising arrangement comprises a sterilising device that operates at ambient pressure.

In an embodiment, said sterilising device comprises a hydrogen peroxide sterilising device. Owing to this aspect of the invention, it is possible to make a sterilising apparatus for treating objects that is compact and always operates at ambient pressure, i.e. for the operation of which it is not necessary to vary the pressure to which the objects to be sterilised are subjected, this enabling an extremely economical apparatus to be obtained.

In a fourth aspect of the invention, a method is provided for treating objects comprising transporting along a curved path said objects by means of a rotating carousel arrangement comprising at least a carousel, sterilising said objects during said transporting with a sterilising arrangement arranged on said carousel arrangement, wherein said sterilising comprises sterilising at ambient pressure.

In an embodiment, said sterilising comprises sterilising by means of hydrogen peroxide.

In a fifth aspect of the invention, a sterilising apparatus is provided for sterilising objects, comprising a conduit for conveying a sterilising solution to said objects, a resistor arranged for heating said solution flowing in said conduit, wherein said apparatus further comprises a heat-accumulating device arranged for accumulating the heat released by said resistor and transferring it to said solution.

In this way the heat-accumulating device accumulates heat transferred by the resistor for a relatively long time, whilst the sterilising solution is heated in a short time, i.e. in the time in which it flows to the objects to be sterilised, this time being defined as injection time.

In an embodiment, a vaporisation chamber is further provided arranged for momentaneously housing said sterilising solution.

In this way the heat-accumulating device accumulates heat transferred by the resistor and simultaneously transfers it to the sterilising solution present in the vaporisation chamber, thus the solution is heated over a relatively long period of time, such period of time being different from the injection time and prior thereto, and the solution is released already in vapour form, during the injection time, flowing to the objects to be sterilised.

In sixth aspect of the invention, a method is provided for sterilising objects by means of a sterilising solution, comprising conveying said solution to said objects, absorbing energy by means of a resistor, wherein there is provided for accumulating said heat produced by said resistor and transferring it to said solution by means of a heat-accumulating device.

In an embodiment, said heat is transferred by said accumulation device to said flowing solution during said conveying.

In this way, the sterilising solution is heated during the time in which it flows along said conduit to said objects to be sterilised, said time being defined as injection time.

In another embodiment, said heat is transferred by said accumulation device to said solution momentaneously housed in a vaporisation chamber provided inside said accumulation device.

In this way, the sterilising solution is heated in a period of time that is different from the injection time and precedes it, and is released already in vapour form, during the injection time, flowing to said objects to be sterilised.

Owing to these aspects, it is possible to heat in a simple and effective manner a sterilising solution that has to be injected in vapour state into objects to be sterilised, these objects being in particular positioned as closely as possible to the heating apparatus.

Apparatuses characterised by high productivity are further obtained.

In a seventh aspect of the invention, a dosing apparatus is provided for dosing a solution, in particular a sterilising solution for sterilising objects, comprising an accumulating chamber arrangement for accumulating said solution, a propelling device for propelling said solution along piping, wherein said apparatus further comprises a pressure-reducing arrangement positioned downstream of said accumulating chamber arrangement and arranged for reducing the pressure of said sterilising solution.

In an embodiment, a flow-intercepting device is provided for intercepting the flow that passes through the pipe.

In an eighth aspect of the invention, there is provided a method for dosing a solution, in particular a sterilising solution for sterilising packaging units, comprising accumulating said solution in an accumulating chamber arrangement, successively propelling said solution along piping, wherein there is provided for reducing the pressure of said solution by means of a pressure-reducing arrangement positioned downstream of said accumulating chamber arrangement.

This enables losing fractions of the sterilising solution along said piping to be avoided, and in particular losing fractions of the sterilising solution in the portion comprised between the chamber arrangement and a heating device of said solution to be avoided.

The invention can be better understood and implemented with reference to the enclosed drawings that illustrate some embodiments thereof by way of non-limitative example, in which:

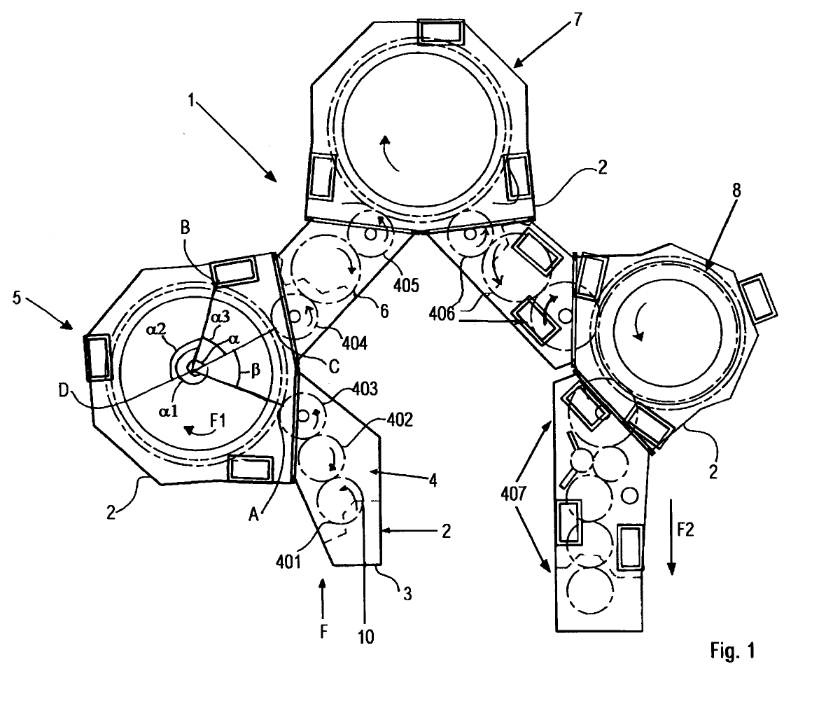

FIG. 1 is a plan view of a first embodiment of an apparatus for treating and filling components of packaging units;

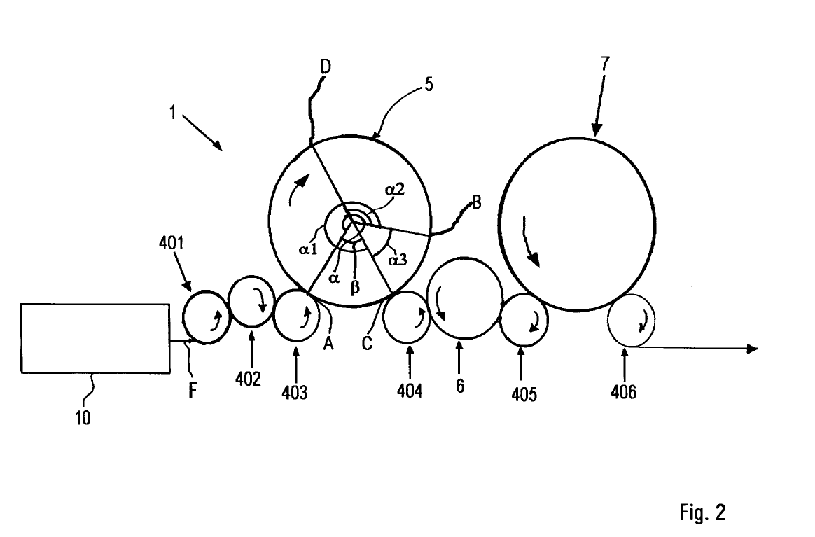

FIG. 2 is a schematic view of the apparatus in FIG. 1;

FIG. 3 is a partial schematic view of a second embodiment of a treatment apparatus for treating components of packaging units;

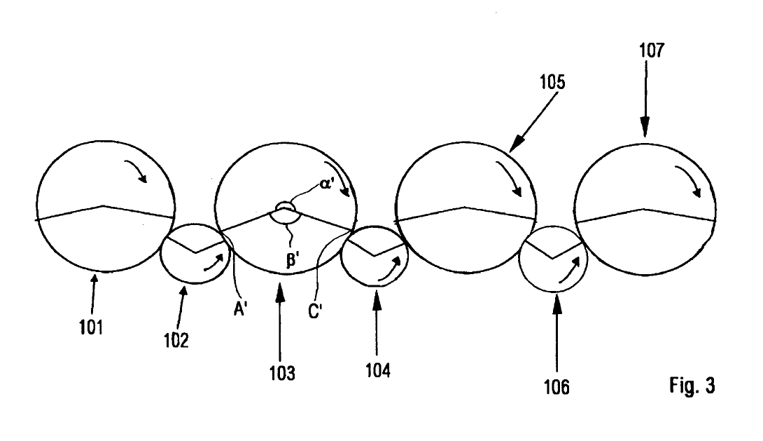

FIG. 4 is a partial schematic view of a third embodiment of a treatment apparatus for treating components of packaging units;

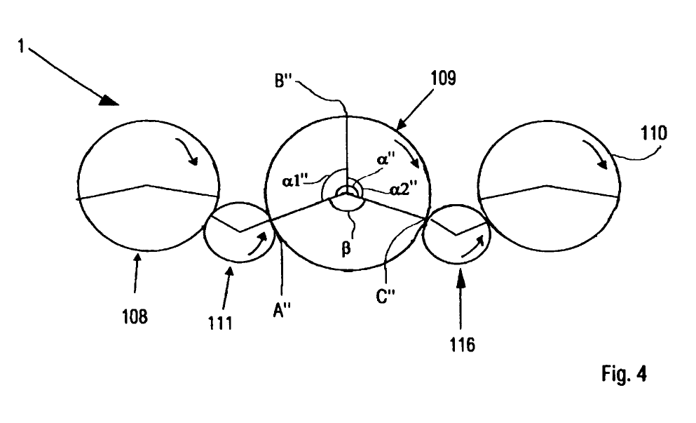

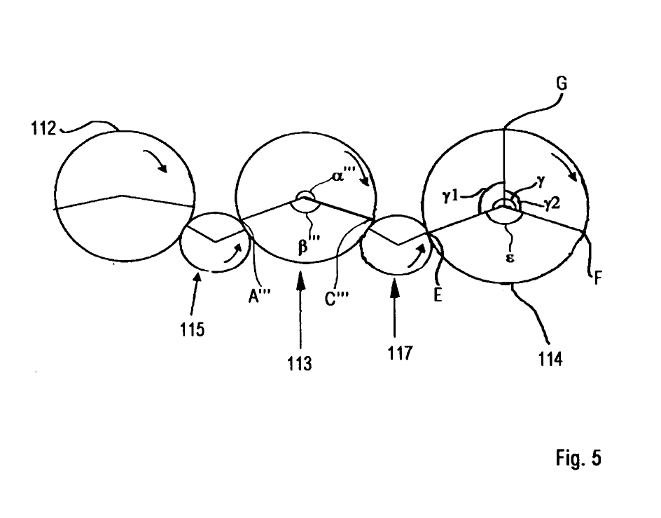

FIG. 5 is a partial schematic view of a fourth embodiment of a treatment apparatus for treating components of packaging units;

FIG. 6 is a partial schematic view of a version of the apparatus in FIGS. 1 and 2;

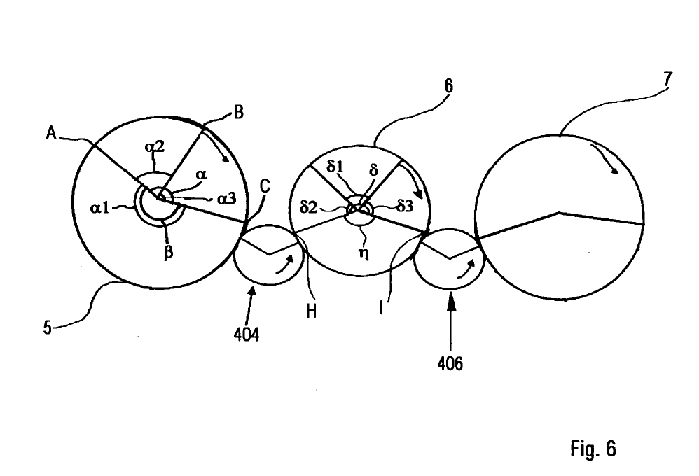

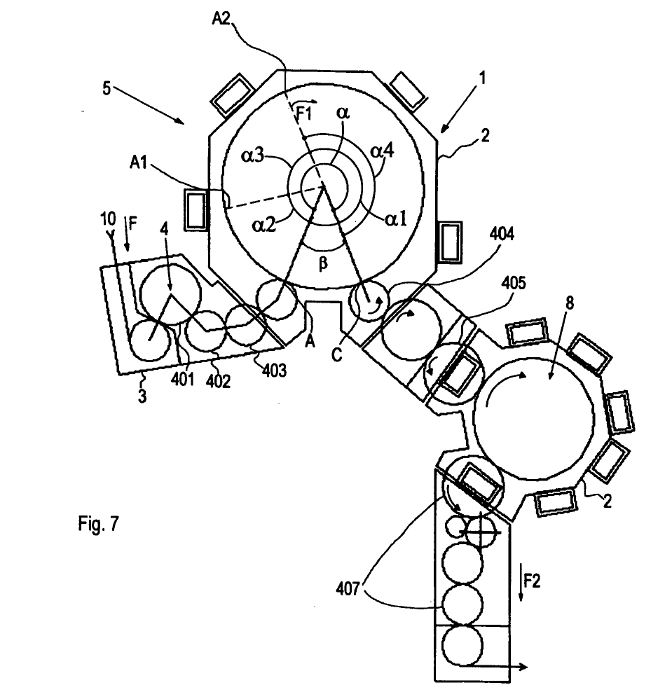

FIG. 7 is a schematic view of a further embodiment of an apparatus for sterilising objects;

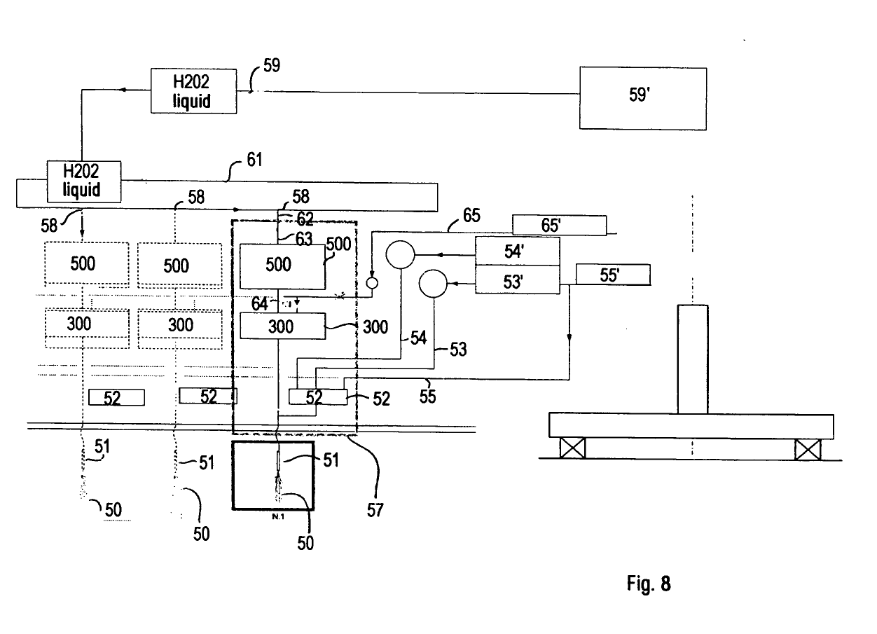

FIG. 8 is a possible diagram that illustrates schematically the devices provided in the treatment carousel in FIGS. 1 and 2;

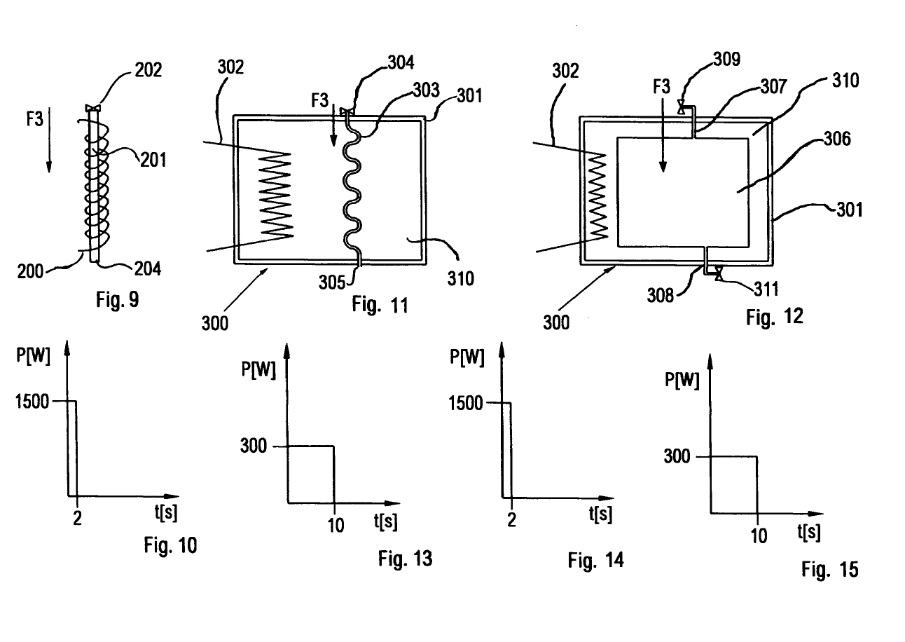

FIG. 9 is a schematic view is a heating system for heating a sterilising solution according to the prior art;

FIG. 10 is a graph that shows the trend over time of the electric power absorbed by the heating system in FIG. 8;

FIG. 11 is a schematic view of a first embodiment of a heating and heat-accumulation system for a sterilising solution according to the invention;

FIG. 12 is a schematic view of a second embodiment of a heating system, of the batch type, of a sterilising solution according to the invention;

FIG. 13 is a graph that shows the trend over time of the of the electric power absorbed by the heating systems for heating the sterilising solution according to the invention, shown in FIGS. 11 and 12;

FIG. 14 is a graph that shows the trend over time of the thermal power transferred by the heating system in FIG. 11;

FIG. 15 is a graph that shows the trend over time of the thermal power transferred by the heating system in FIG. 12;

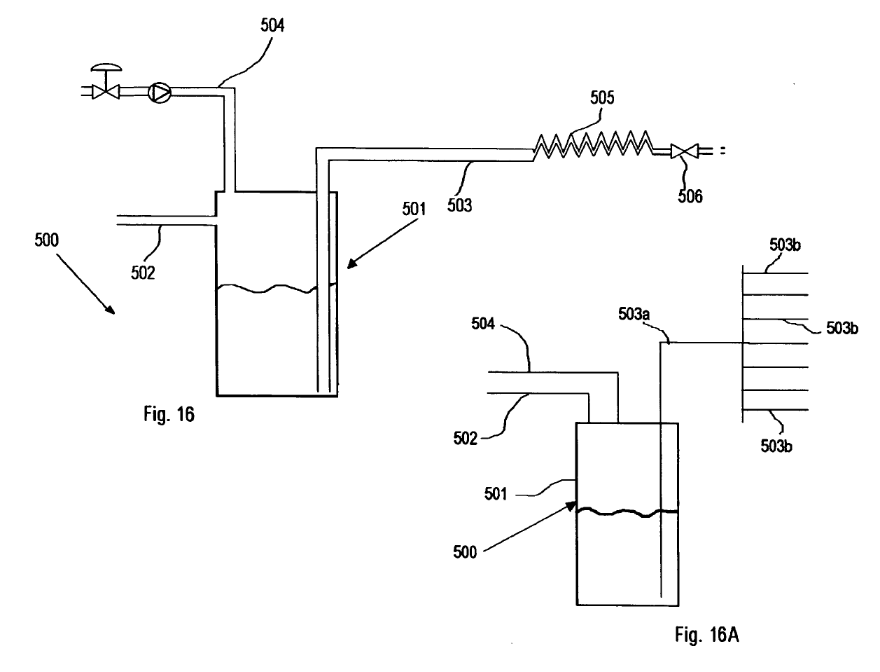

FIG. 16 is a schematic view of an embodiment of a dosing system for dosing the sterilising solution according to the invention;

FIG. 16A is a schematic view of the structure of the dosing system for dosing the sterilising solution of FIG. 16;

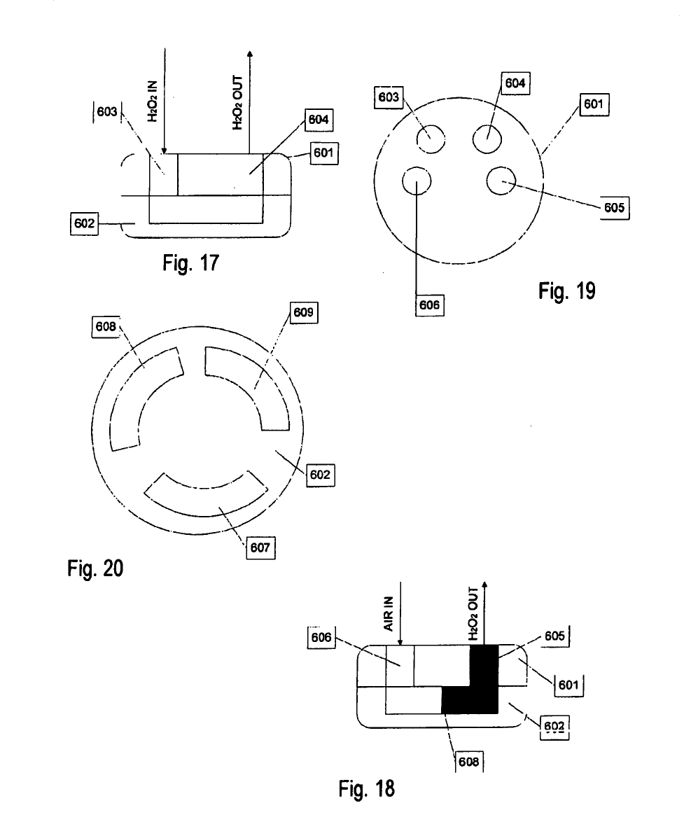

FIG. 17 is a schematic section of a further embodiment of a dosing system for dosing the sterilising solution according to the invention in a first operating phase;

FIG. 18 is a schematic section of the dosing system in FIG. 17 in a further operating phase;

FIG. 19 is a schematic view from above of a first disc of the dosing system in FIG. 17;

FIG. 20 is a schematic view from above of a second disc of the dosing system in FIG. 17;

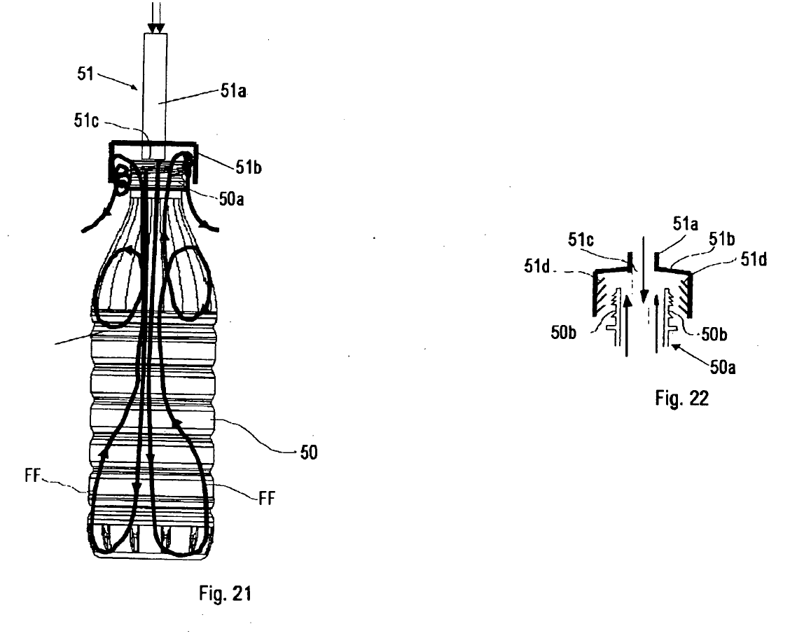

FIG. 21 is a schematic view of a sterilising nozzle for sterilising containers that shows the circulation of a sterilising solution;

FIG. 22 is an enlarged detail of the nozzle in FIG. 21.

With reference to FIGS. 1 and 2, there is shown a system 1 for treating components of packaging units coming from a supplying line 10, in particular of bottles and/or caps suitable for being used for obtaining filled sterile containers.

The apparatus 1 is externally delimited by walls 2 having the function of insulating it from the external environment, to ensure that aseptic conditions are maintained in the interior thereof. For this purpose, inside the walls 2 slight overpressure can be generated with respect to the external environment to prevent the entry of air and thus of possible contaminants from the external environment.

The bottles to be sterilised are introduced into the apparatus 1 from the supplying line 10 in the direction of the arrow F in the inlet zone 3 at a first sterilising zone 4 and are subjected to a series of treatments along their path inside the apparatus 1, in particular, they are subjected to the action of a plurality of sterilising devices provided along this path.

In the first sterilising zone 4, a first transporting star 401, a second transporting star 402 and a third transporting star 403 are provided that are rotatable around the respective vertical axes in the direction indicated by the respective arrows, and interacting between themselves to shift the bottles from the supplying line 10 to an inlet A of a carousel 5 along a sinuous path that passes through sterilising devices intended for sterilising the external surface of the bottles.

The carousel 5 draws the bottles by means of a drawing arrangement that are not shown and comprising, for example, drawing grippers, that grasp the bottles during the entire journey thereof on the carousel 5. The carousel, rotating in the direction indicated by the arrow F1, rotates the bottles, each one of which is positioned at a treatment station, defined, for example, by a pair of drawing grippers, in suitable treatment zones provided on the carousel 5 and provided in succession.

The treatment zones are defined as follows.

The bottles that enter the carousel 5 from the inlet A are taken by the rotating carousel to an outlet C at which the bottles are removed from the carousel 5 and move to a fourth transport star 404 that takes them to a cooling carousel 6.

Between the inlet A and the outlet C an active zone α is defined in which the various devices provided on the carousel 5 interact with the bottles, whilst between the outlet C and the inlet A a passive angular sector is defined, known also as a “dead angle” along which the carousel 5 is devoid of bottles.

Between the outlet C and a point B, a heat accumulation sector α1 is provided, extending along the passive sector β and along a part of the active sector α, in which, depending on the type of heating device for heating the hydrogen peroxide solution provided, different operations occur.

In particular, if the heating device for heating the solution, disclosed below with reference to FIG. 11 has been provided, this device accumulates in a metal material the heat necessary for subsequent vaporisation of the solution, this vaporisation occurs in the portion a3 simultaneously with the injection of the solution into a bottle to be sterilised.

If, on the other hand, the heating device is provided for heating the solution disclosed below with reference to FIG. 12, this device transfers, in the heat accumulating sector α1, the heat necessary for the vaporisation of a dose of solution, to a dose of solution housed in a vaporising chamber. In this case, at the sector α3 the solution is already vaporised and is injected only into a bottle to be sterilised.

In general, whatever the system of vaporisation of the sterilising solution provided, the device provided at a set treatment station of the carousel 5, start to accumulate heat in the accumulation mass and, as in the embodiment in FIG. 12, starts to transfer it to the sterilising solution immediately after the phase of injection of sterilising solution into a bottle in that given treatment station, using in this way all the useful rotation time of the carousel 5.

In a portion of the heat accumulating sector α1, between a further point D and the point B, there is provided a preheating sector α2, in which also the internal surface of the bottles is heated.

Lastly, between the point B and the outlet C a vaporisation and injection sector α3 is provided (as in FIG. 11) or an injection sector for injecting already prepared vapour (as in FIG. 12), is provided, in which the hot hydrogen peroxide solution is injected in vapour state inside the bottles and is left in contact with the internal surface of the bottles so as to bring about the sterilisation reaction.

The previous heating of the internal surface of the bottles prevents the vaporised hydrogen peroxide solution condensing too rapidly in contact with the surface of the bottles and thus not homogeneously spreading over the entire surfaces of the bottles, and further enables rapid activation of the sterilising solution in the zone in contact with the wall and thus the rapid start of the sterilisation reaction.

In fact, in order for the hydrogen peroxide sterilising solution to be activated, it is necessary for the activating energy to be reached, namely for the wall of the bottles to be sterilised to have a temperature of approximately 50°-70° C.

The temperature of the bottles must not, however, bee too high to prevent the sterilising solution evaporating too fast so that it does not remain sufficiently long in contact with the internal surfaces of the bottles, and also to prevent thermal deformation that would change the shape and volume of the bottles.

The position of the further point D and of the point B on the carousel 5 can be suitably varied according to given process requirements for varying the duration of the bottle preheating phase and/or the vaporisation phase of the sterilising solution.

Successively, the bottles are unloaded from the carousel 5 to a fourth transport star 404 that takes them to a cooling carousel 6, in which they are subjected to a stream of cold air at ambient temperature that is suitable for preventing deformation of the bottles due to the excessive temperatures reached and to the contact of the vapours of the sterilising solution with the walls of the bottles.

Subsequently, the bottles are removed by a fifth transport star 405 that takes them to a drying carousel 7, wherein they are subjected to a stream of dry hot air suitable for evaporating the hydrogen peroxide solution present in the bottles, in order to eliminate any residue of the solution that could compromise subsequent use of the bottles.

Subsequently, by means of further transport stars 406, the sterile bottles are supplied to further treatment phases, for example to a filling carousel 8, which is also placed inside the sterile walls 2 in which therefore, always in aseptic conditions, the bottles can be filled with suitable contents and be possibly capped and/or sealed.

From the filling carousel 8 the bottles are evacuated by means of still further transport stars 407 along a sinuous path moving away from the apparatus 1 in the direction indicated by the movement arrow F2.

With reference to FIG. 7, an embodiment of a further apparatus 100 is shown for treating objects, and in particular bottles, this apparatus comprises some parts having the same function and/or structure, and/or in which the same operations of corresponding parts of the apparatus 1 occur, which will therefore be indicated by the same numeric references and will not be explained in detail.

Bottles to be sterilised are moved along the supplying line 10 in the direction of the arrow F and introduced into the apparatus 100 in the inlet zone 3 at a first sterilising zone 4 in which, by means of a plurality of transport stars, the bottles are moved to an inlet A of a carousel 5′ along a sinuous path that passes through sterilising devices intended for sterilising the external surface of the bottles.

The carousel 5′ by rotating in the direction indicated by the arrow F1, transports the bottles as it rotates, each of which bottles is positioned at a treatment station, defined, for example, by a pair of drawing grippers, in suitable treatment zones provided on the carousel 5 and arranged in succession.

The treatment zones of the carousel 5′ are those provided in the carousel 5 of the apparatus 1 and differ from the latter as in the carousel 5′ a drying zone is also provided as explained better below.

The bottles that enter the carousel 5′ from the inlet A are taken by the rotating carousel to an outlet C at which the bottles are removed from the carousel 5′ and move to a fourth transport star 404.

Between the inlet A and the outlet C an active zone α is defined in which the various devices provided on the carousel 5′ interact with the bottles, whereas between the outlet C and the inlet A a passive angular sector β is defined, also known as a “dead angle” along which the carousel 5 is devoid of bottles.

Between the point A2 and the point A1 a heat accumulating sector α1 is provided, extending along the passive sector β and along a part of the active sector α, in which, depending on the type of heating device provided for heating the hydrogen peroxide solution, different operations occur, as already seen with reference to FIG. 1.

In general, whatever vaporisation system of the sterilising solution is provided, the device provided at a given treatment station of the carousel 5′ starts to accumulate heat in the accumulation mass and, embodiment of FIG. 12, to transfer it to the sterilising solution immediately after the phase of injection of sterilising solution into a bottle in that given treatment station has terminated, using in this way for accumulating heat, all the useful rotating time of the carousel 5′.

In a portion of the heat accumulating sector α1, between inlet A and point A1, there is provided a preheating sector α2, in which heating the internal surface of the bottles is also provided.

Between point A1 and point A2 a vaporisation and injection sector α3 is provided (as in FIG. 11) or injection sector for injecting an already prepared vapour is provided (as in FIG. 12), in which the hot hydrogen peroxide solution is injected in vapour state inside the bottles and is left in contact with the internal surface of the bottles so as to bring about the sterilisation reaction.

Lastly, in a portion of the accumulating sector α1, between point A2 and the inlet A, a drying sector α4 is provided, in which the bottles are subjected to a stream of dry hot air suitable for evaporating the hydrogen peroxide solution in the bottles, in order to eliminate any residue of this solution that could compromise the subsequent uses of the bottles.

The position of the further point A1 and of the point A2 on the carousel 5′ can be appropriately varied on the basis of given process requests for varying the duration of the preheating phase of the bottles and/or the vaporisation phase of the sterilising solution.

Subsequently, the bottles are unloaded from carousel 5′ to the transport stars 420 that by means of sinuous paths take the bottle to possible other treatment carousels and/or treatment devices that may, for example, comprise, a cooling carousel, or a filling carousel 8, as shown in FIG. 7, etc.

With reference to FIG. 3 a second embodiment of the treatment apparatus 1 is shown schematically, comprising a preheating carousel 101 that receives the bottles to be sterilised coming from a first sterilising zone that is not shown in which the external surface of the bottles was sterilised.

From the preheating carousel 101 the bottles are drawn by a first transport star 102 that takes them to a treatment carousel 103, in which each bottle is treated with a given quantity of a hydrogen peroxide sterilising solution in vapour state to sterilise the internal surface thereof.

In the treatment carousel 103 there is defined an inlet A′ at which the bottles are introduced into the treatment carousel 103 and an outlet C′ at which the bottles are evacuated from the treatment carousel 103 and move to a second transport star 104 that takes them to a cooling carousel 105.

Between the inlet A′ and the outlet C′ there is defined an active zone α′ in which the various devices provided on the treatment carousel 103 interact with the bottles, in particular the overheated vapours of the hydrogen peroxide solution are injected inside each bottle and interact with the internal surface of the latter to sterilise it, whilst between the outlet C′ and the inlet A′ there is defined a passive angular sector p′, along which the carousel 103 is devoid of bottles.

Also in this case, the same considerations apply to the vaporisation systems for the sterilising solution provided as apply to the embodiment in FIG. 1 with reference to two embodiments of the heat accumulating device in FIGS. 11 and 12.

In the cooling carousel 105 the bottles are subjected to a stream of cold, sterile and dry air. Subsequently, by means of a third transport star 106, the bottles are removed from the cooling carousel 105 and taken to a drying carousel 107 in which they are treated with a stream of hot and sterile dry air to evaporate the residues of hydrogen peroxide.

With reference to FIG. 4 a third embodiment of the apparatus in FIG. 1 is presented that differs from the embodiment in FIG. 3 through the fact that treating the bottles with hydrogen peroxide solution and cooling them are carried out in the same carousel.

In this embodiment the apparatus comprises a preheating carousel 108, a treatment and cooling carousel 109, and a drying carousel 110 between which the bottles are moved in succession by means of suitable transport stars 111, 116.

In the treatment and cooling carousel 109 there is defined an inlet A″ at which the bottles are introduced inside the treatment and cooling carousel 109 and an outlet C″.

Between the outlet C″ and the inlet A″ there is defined a passive angular sector β″, along which the carousel 109 is devoid of the bottles and in which the hydrogen peroxide solution is preheated and is then injected into the bottles in vapour state. Between the inlet A″ and the outlet C″ there is defined an active zone α″ comprising a treatment sector α1″, extending between the inlet A″ and a point B″, in which the hydrogen peroxide solution is injected inside each bottle, and a cooling sector α2″, extending between point B″ and the outlet C″ in which the bottles are cooled.

The position of point B″, and therefore the duration respectively of the treatment phase and of the heating phase can be varied appropriately on the basis of specific process requests.

Also in this case, the same considerations apply to the operation of the vaporisation systems of the sterilising solution provided as apply to the embodiment in FIG. 1 with reference to two embodiments of the heat accumulating device in FIGS. 11 and 12.

With reference to FIG. 5 a fourth embodiment of the apparatus in FIG. 1 is presented, that differs from the embodiment in FIG. 3 by the fact that cooling and drying of the bottles are conducted in the same carousel.

In this embodiment, the apparatus comprises a preheating carousel 112, a treatment carousel 113, and a cooling and drying carousel 114 between which the bottles are transported in succession by means of appropriate transport stars 115, 117.

In the treatment carousel 113 an inlet A′″, an outlet C′″ and the same sectors are provided, marked respectively as a′″ and β′″, provided in the carousel 103, for which the same considerations apply to the vaporisation systems for the sterilising solution provided as apply to the embodiment FIG. 1.

In the cooling and drying carousel 114 there is defined an inlet E and an outlet E′ between which there is defined an active angular sector γ comprising a cooling sector γ1 extending between the inlet E and a point G the position of which can be varied on the basis of process requirements, in which the bottles are cooled, and a drying sector γ2 extending between the point G and the outlet F in which the bottles are dried. Between the outlet F and the inlet E there is defined a passive angular sector ε in which the cooling and drying carousel 114 is devoid of bottles.

With reference to FIG. 6 an embodiment of the apparatus in FIG. 1 is presented, in which the same devices are defined by the same alphanumeric references and which comprises a cooling carousel 6 in which there is provided for varying the duration of the cooling operation.

In the cooling carousel 6 there is defined an inlet H and an outlet I, between which an active sector δ is defined in which the cooling carousel 6 is provided with bottles to be cooled, and which comprises an effectively active sector δ1 in which cooling actually occurs, that is the bottles are treated with a stream of cold cooling air in the effectively active sector δ1. The active sector δ further comprises transporting sectors δ2 and δ3 in which the bottles are not subjected to any treatment, whilst between the outlet I and the inlet H, a passive sector η is defined in which the carousel 6 is devoid of bottles.

The width of the active sector δ1 and of the transporting sectors δ2 and δ3 can be reciprocally varied in an appropriate manner on the basis of particular process requirements.

As can be seen, the different embodiments shown of treatment apparatuses for treating containers, and in particular bottles, enable the bottles to be sterilised in an effective manner with hydrogen peroxide at ambient pressure, i.e. without the pressure of the bottles and/or the operating pressure of the treatment devices of the bottles along the apparatuses being varied along the path of the bottles along the apparatuses.

The apparatuses disclosed above can further sterilise the bottles also with sterilising agents other than hydrogen peroxide.

With reference to FIG. 8 a possible diagram is presented that illustrates schematically some devices provided in a treatment carousel of parts of components of packaging units, for example in the carousel 5 of FIGS. 1 and 2.

The carousel 5 is peripherally provided with a plurality of positioning and gripping elements, that are not shown, comprising for example a plurality of pairs of grippers, for gripping components of packaging units, for example of bottles 50, such plurality of pairs of grippers draw the bottles 50 that have to be treated and keep them in position on their path on the treatment carousels, for example on carousel 5.

This plurality di positioning and gripping elements, defines on the carousel 5 a plurality of treatment stations for treating the bottles 50.

Each station is provided with a nozzle 51 that is movable in a manner that is integral with the station, shown in greater detail in FIGS. 21 and 22, and that can be connected, by means of a distributing element 52, alternatively to a supplying line of hot air 53, coming from a corresponding accumulating zone for accumulating preheating hot air 53′ and suitable for heating the bottles 50 in the preheating phase, (sector α2), with a further supplying line of hot air 54, coming from a corresponding accumulating zone 54′ of hot air and suitable for supplying hot air that is mixed with the vaporised hydrogen peroxide solution to be introduced into the bottles 50 in the treatment phase, (sector α3), and with a supplying line of sterile air at ambient temperature 55, supplied by a corresponding accumulating zone of sterile air at low pressure 55′, for cooling the bottles 50.

The nozzle 51 may further be connected during the bottles 50 treatment phase to a conduit 56 to enable the vaporised sterilising solution enter inside each bottle 50.

With reference to FIGS. 21 and 22, the nozzle 51 comprises a cylindrical body 51a into which flows, during the treatment phase, the vapours of the hydrogen peroxide sterilising solution for treating the bottle 50 and a hat-shaped portion 51b arranged for surrounding a portion of neck 50a of the bottle to be sterilised and provided with flaps 51d.

The shape of the nozzle 51 seen above enables all the parts of the bottle 50 to be treated in an effective manner, and in particular enables the external surfaces 50b of the neck portions 50a of the bottles 50 to be sterilised.

The neck of the bottle and, in particular, the thread on which the cap of the bottle is screwed is a very ticklish zone that has to be sterilised appropriately, also because a user may consume the contents of the bottle by drinking it directly from the bottle, and on the other hand it must be completely devoid of residues of sterilising solution that could be harmful for a user.

Thus whilst the external surface of the body of the bottles can be sterilised with equanimity, for example with a sterilising solution of hydrogen peroxide in liquid state, without excessively worrying about possible residues of solution on the surface of the body of the bottle, or possibly can be treated in the same way in which the internal surface of the bottle is treated, to the neck the same restrictions apply as those seen for the internal surface of the bottles to be sterilised, and they therefore have to be treated with hydrogen peroxide vapours.

The hydrogen peroxide sterilising solution vapours are injected into bottle to be sterilised from a port 51c of the nozzle 51 and flow inside the bottle 50, as shown by the flow lines FF in FIG. 21, reaching the bottom of the bottle 50, touching the internal surfaces thereof and ascending again to the neck 50a to then exit the bottle 50.

If the nozzle 51 was devoid of the hat portion 51b, the hydrogen peroxide vapours would flow, after sterilising the inside of the bottle 50, outside the bottle 50 in a non-controlled manner, risking not sterilising in an appropriate manner the external surface 50b of the neck portion 50a of the bottles 50.

The presence, on the other hand, of the hat portion 51b and of the flaps 51d, force the sterilising solution vapours to flow with a turbulent and spiraling flow so that they touch the external surface of the neck portion 50b and the thread thereof, sterilising it in an effective manner before flowing freely outside the bottle 50.

At each station a pretreatment system for pretreating the sterilising solution is further provided that is overall indicated by 57, comprising a dosing system for dosing the solution 500 that is disclosed in greater detail with reference to FIG. 14, and a heating system for heating the solution 300 that is disclosed in greater detail with reference to FIGS. 10 and 11 and that is positioned downstream of the dosing system and which supplies the conduit 56 with vaporised solution.

Each dosing system 500 for dosing the solution receives liquid hydrogen peroxide solution from a respective branch 58 of a supplying line of the liquid solution 59, which in turn receives it from a storage tank 59′ for storing the solution; the branch 58 comprises a loop circuit zone 61 to keep the solution circulating in order to prevent the formation of gas within it and to enable it to be renewed continuously in the pipes.

The liquid solution, therefore, from the branch 58, by means of the conduit 62 provided with an opening/closing valve 63, is introduced into the dosing system 500, from the latter a properly determined dose of solution passes through the further conduit 64 to the heating system 300, from which the vaporised solution, passes through the conduit 56 into the nozzle 51 and then into the bottles 50.

Sterile air is further introduced into the heating system 300 by means of the line 65 connected to a corresponding accumulating zone 65′ for accumulating sterile air, sterile air being used as a transporting fluid for the hydrogen peroxide vaporised solution, in order to prevent residues of solution remaining inside the heating system 300 and/or inside the conduit 56 and thus do not reach the bottle 50 so that sterilisation is thus not effective.

With reference to FIG. 9, heating system is shown for heating a sterilising solution according to the prior art.

For sterilising the containers, a vaporised sterilising solution, for example a hydrogen peroxide solution is injected inside them.

This solution is normally stored in liquid state and has to be vaporised along its path between the storage tank and the point of injection into the bottles.

In order to obtain the aforementioned heating, in the prior art, as shown in FIG. 9, each conduit 201 that conveys the sterilising solution to the bottles present on a sterilising machine is provided with an electric resistor 200 that is wound around at least a part of this conduit.

When at the conduit 201 there is a bottle 50 to be sterilised a valve 202 is opened that is located along the conduit 201, so that a given quantity of sterilising solution is intake into the conduit 201 flows inside the latter, as shown by the flow arrow F3.

Simultaneously, the resistor 200 connected by means of appropriate transformers that are not shown to the electric network, absorbs power from the electric network, so heating almost instantaneously and simultaneously transferring the heat to the solution that flows in the conduit 201.

In this way, the solution enters the conduit 201 by means of the valve 202 in liquid state and exits therefrom, at the end 204 placed on the part of the bottle to be sterilised, in vapour status.

In the graph in FIG. 10 the trend over time [s] of the power absorbed [W] by the resistor 200 is shown.

As can be seen from this graph, for each resistor total energy is absorbed from the electric network amounting to about 3000 Joule, over a time of about 2 sec, which is the sterilising time, i.e. the time that elapses between the moment in which a determined bottle is loaded onto a sterilising machine and the moment in which vaporisation of the solution is obtained inside the bottle, the total energy absorbed is equivalent of instant absorbed power of 1500 W.

By installing such a vaporising device for each of the treatment stations provided in a treatment machine, for example of the rotating carousel type, the following would occur:
all the vaporisers mounted on the treatment carousel (i.e. not only those present at a given instant in the active sector α3) have to be switched on simultaneously;
control of the resistances is performed using the PWM method, i.e. the modulation of electric power is achieved by alternating instants of maximum power with instant of zero power.

Therefore, the condition would arise in which in some instants the total electric power required for the operation of the machine is the same as the product of the installed power of a single vaporiser for the total number of the stations provided in the aforementioned machine.

In a sterilising carousel of large dimensions, in which the number of stations is high, for example 80 or also more, an installation of the type seen above would thus require very high installed power to be transmitted on the rotating part, with the constructional and structural limitations seen previously.

These drawbacks are overcome by a heating system of the sterilising solution according to the invention of which, with reference to FIGS. 11 and 12, two embodiments are shown, the heating system uses a heat accumulation mass for accumulating the heat transferred by the resistor and then transferring it to the sterilising solution.

In the embodiment in FIG. 11, the heating system 300 comprises a heat accumulator 310 of material with high thermal capacity, i.e. in a material capable of accumulating a great quantity of heat within the mass thereof, which is enclosed inside a layer of insulating material 301, and within which a resistor 302 is immersed, connected, by means of appropriate transformers that are not shown, to the electric network.

In the heat accumulator 310 a conduit 303 is furthermore obtained that is provided with a valve 304 positioned on the side of the further conduit 64, the conduit 303 communicates at one end 305 thereof located on the side of the conduit 56 with a bottle to be sterilised.

The operation of this system is as follows: at each cycle, immediately after a given bottle has been unloaded from the treatment carousel, i.e. when a given treatment station is in the position C in FIGS. 1 and 2, the resistor 302 starts to transfer energy to the heat accumulator 310, which continues to absorb it until the aforementioned treatment station, into which in the meantime a bottle 50 has been loaded in position A of FIGS. 1 and 2, is in the treatment start position, for example in position B in FIGS. 1 and 2.

In position B, the valve 304 is opened so that the solution flows into the conduit 303, as indicated by the flow arrow F3, the solution enters the conduit in liquid state, absorbs the heat that is transferred to it by the mass 310 and exits from the end 305 in vapour state flowing into conduit 56 and then into the bottle 50 to be sterilised.

In this system the heat accumulator 310 absorbs energy from the resistor 302 for a relatively long time, lasting approximately 10 seconds, corresponding, for example with reference to FIGS. 1 and 2, to the width of the rotation arc α1, and transfers this energy to the solution in a short length of time, approximately 2 seconds, as shown in FIGS. 13 and 14 respectively.

In the embodiment in FIG. 12, the system comprises a heat accumulator 310 inside which a vaporising chamber 306 is created that is connected by means of portions of conduit 307, 308, respectively to a first valve 309 connected to the further conduit 64 and to a second valve 311 connected to the conduit 56.

The operation of such system is as follows: at each cycle, immediately after a given bottle has been unloaded from the treatment carousel, the first valve 309 is opened, keeping the second valve 311 shut, so that a given quantity of liquid sterilising solution is loaded in the vaporising chamber 306, simultaneously, after the shutting of the valve 309, the resistor 302 starts to transfer energy, by means of the heat accumulator 310, to the solution in the vaporising chamber 306 making it evaporate before the treatment station, at which a bottle 50 has in the meantime been loaded, is in the treatment start position.

When the treatment station is in the treatment start position the second valve 311 is opened in such a way that the solution, already in vapour state flows therefrom, and is injected into the bottle 50 to be sterilised, by means of the pressure that is created inside the vaporising chamber 306.

In this system, the heat accumulator 310 absorbs energy from the resistor 302 and simultaneously transfers this energy to the solution housed in the vaporising chamber 306 for a relatively long time, lasting approximately 10 seconds, corresponding, for example with reference to FIGS. 1 and 2, to the width of the rotation arc α1, as shown in FIG. 15, and in the treatment phase the solution is already in vapour state ready to be injected into the bottle 50.

In the graphs in FIGS. 13 and 14 there is shown respectively the trend of the absorbed power and of the transferred power transferred to the fluid by the heating system for heating the sterilising solution according to the invention in the embodiment in FIG. 11.

In the graph in FIG. 15 there is shown the trend of the electric power absorbed by the resistance and of the thermal power transferred to the fluid by the heating system for heating the sterilising solution of FIG. 12.

As can be seen from the analysis of these graphs, these systems use an inactive “long” phase of work, i.e. a phase in which injection of the vapour of the hydrogen peroxide sterilising solution H₂O₂ into the bottle to be sterilised does not occur, but in which the resistor 302 absorbs low-power electric energy for a relatively long time, for example: 300 W for 10 sec=3000 joule, reducing significantly in this way, the total electric power to be installed.

As already said, this enables significant electrical wiring problems to be eliminated and treatment carousels to be installed that are provided with a great number of treatment stations.

These systems further provide an active “short” operating phase in which the injection of the previously vaporised hydrogen peroxide sterilising solution occurs; in the latter phase on the other hand the following conditions obtain:
in the case of the embodiment of the heating system in FIG. 11, for a time equal to 2 sec the material of the heat accumulator 310 transfers to the sterilising solution the 3000 joule that had been previously accumulated, at a thermal power equal to 1500 W;
in the case of the embodiment of the heating system in FIG. 12, the sterilising solution is already in vapour state and is ready and available for injection, injection lasting approximately 2 seconds.

As can be seen from the analysis of this graphs, these systems increase, with respect to known systems, the duration of the energy absorption phase by the resistor 302 from 2 seconds to 10 seconds, so for the same total energy absorbed, the instantaneous absorbed power become equal to approximately 300 W with respect to the 1500 Watt of power absorbed by the prior-art system. Such energy being accumulated in the form of heat, in a heat accumulation mass (FIG. 11), or, by means of a heat accumulation mass in a given quantity of sterilising solution.

With reference to FIGS. 16 and 17 to 20 two embodiments are shown in detail of the dosing system 500 of the sterilising solution to be positioned, for example, upstream of the heating system 300 of the solution.

With reference to FIG. 16, the dosing system 500 comprises an accumulation chamber for accumulating the sterilising solution 501 connected to a first pipe 502, connected to the conduit 62 to supply the pressurised liquid solution of H₂O₂ inside the accumulation chamber 501, a second pipe 503 arranged for conveying the sterilising solution outside the accumulation chamber 501 and supplying it, by means of the further conduit 64 to the heating system 300 of the solution, and a third pipe 504, connected to a source of compressed air and to a movement pump 507, and arranged for supplying compressed air to the accumulation chamber 501 for propelling the solution into the second pipe 503.

The pipe 503 comprises, as shown by FIG. 16A, a main portion 503a connected to the accumulation chamber 501 from which a plurality of pipes 503b leads away, shaped in such a way that each treatment station provided in the treatment carousel is supplied by a corresponding pipe 503b.

On each of these pipes 503b a device is provided for creating a flow resistance, such device comprising for example a capillary pipe 505 having a section that is reduced with respect to the section of the pipe 503b and a valve 506 of the on/off type that is movable between a closed position in which the valve 506 blocks the passage of the solution in the pipe 503b, and an open position in which the valve 506 enables the passage of the solution in the pipe 503b towards one of the heating systems 300 for heating the solution previously discussed.

The valve 506 has a very limited internal volume, which is negligible with respect to the volume of sterilising solution to be dosed at each cycle, the volume being comprised for example between 30 and 100 μl.

The dosing system 500 enables to dose with extreme precision the doses of sterilising solution that have to be sent to each heating system 300 and therefore to each bottle 50, this provides the certainty of vaporising completely the dose of solution and therefore to effectively sterilise each bottle to be treated.

The operation of the dosing system 500 is of proportional type in function of parameters such as the length of the opening time of the valve 506 and the pressure of the solution in the pipe 503.

When the valve 506 opens, i.e. when a dose of solution has to be supplied to a heating system 300 for heating the solution, the pressure of the solution pushes the solution to flow from the chamber 500 through the pipe 503 and through one of the pipes 503b, through the capillary pipe 505 and the valve 506 that remains open until it is traversed by a quantity of sterilising solution that is the equivalent of a dose, after which the valve 506 shuts.

This quantity of solution or dose of solution is measured in time, i.e. when the pressure of the solution upstream of the capillary pipe 505 is known, i.e. the pressure in the third pipe 504 is known, and when the pressure downstream of the capillary pipe 505 is known, i.e. when the flow resistances are known to which the solution is subjected along the capillary pipe 505, the flow of the solution and therefore the quantity of solution that has flowed through the valve 506 in a set period of time are known.

To assure that the dosing system is precise the pressure of the solution must be as constant as possible and the solution must have high pressure, approximately 1 bar.

A dose of solution is usually a quantity comprised between 0.3 and 1 ml, depending on the features of the container, and/or on the closure of the container to be sterilised, and is therefore a very small quantity.

For dosing in a precise manner these small quantities of solution great flow resistance are necessary, i.e. it is necessary to make piping with very small passage diameters of a few tenths of a millimetre.

This constitutes a problem due to the likelihood of clogging of pipes with such reduced dimensions during operation in a continuously operative industrial environment.

In order to overcome the above drawbacks, the invention provides for transforming the concentrated flow resistances obtained with small diameters into an equivalent distributed flow resistance distributed along the capillary pipe 505 that has a length L, for example comprised between 10 and 100 cm, and a diameter that is greater than the diameter required to achieve the required concentrated flow resistances, i.e. a diameter greater than 0.5 mm so as to avoid or minimise clogging phenomena.

With reference to FIGS. 17 to 20, a second embodiment is shown of a dosing system 600 for dosing the sterilising solution to be positioned upstream of the heating system 300 of the solution.

The dosing system 600 comprises a first pipe for supplying the pressurised liquid sterilising solution of H₂O₂ to a second pipe 603 comprising a common portion common to all the treatment stations of the same carousel, from which a plurality of pipes leads away, one for each treatment station provided on the carousel and each provided with the dosing system 600 and a third piping connected to a source of compressed air.

The dosing system 600 comprises a first disc 601 and a second disc 602 superimposed on one another and connected together so that the second disc 602 can rotate with respect to the first disc 601.

In the first disc 601 are made first through holes 603 and 604 respectively for the inlet and the outlet of the liquid hydrogen peroxide sterilising solution (H₂O₂), and second through holes 605 and 606 respectively for the inlet and the outlet of compressed air.

In the second disc 602 a first slot 607, a second slot 608 and a third slot 609 are made that do not go through the second disc 602 but are obtained in the surface thereof.

The dosing system 600 operates in the following manner: the first disc 601 and the second disc 602 are positioned in such a way that the second slot 608 is positioned that the at the ends thereof are situated the first holes 603 and 604, position of FIG. 17, in this way when the liquid sterilising solution of H₂O₂ is dispensed the second slot 608 fills with a known volume of sterilising solution, this volume being determined by the dimension of the slot 608.

The second disc 602 is then rotated so that the second slot 608 and the ends thereof moves to the second holes 605 and 606 in this way enabling the compressed air to evacuate the volume of sterilising solution present in the second slot 608, position of FIG. 18, and to transport it to the heating system 300.

Following this rotation the third slot 609 moves to the first holes 603 and 604, thus getting filled with liquid sterilising solution, up to a known volume determined by the dimension of the slot 609.

The disc 602 is then rotated again, repeating the process disclosed before, namely the third slot 609 is taken to the second holes 605 and 606 so that the compressed air can evacuate the volume of sterilising solution present in the third slot 609.

Thus, each subsequent rotation of the second disc 602 is matched by volumetric dosing of sterilising solution defined by the volume of each slot, and by transporting said defined volume of solution by means of air.

1-278. (canceled)

279. An apparatus for treating objects comprising a plurality of rotating carousels for transporting said objects along a curved path, sterilizing devices arranged on said plurality of carousels along said path, wherein said sterilizing devices are distributed on a carousel of said plurality of carousels and partially on at least a further carousel of said plurality of carousels.

280. An apparatus according to claim 279, wherein said sterilizing devices comprise a first sterilizing device positioned in a first sterilizing zone of said apparatus located upstream of said plurality of carousel arranged for sterilizing an external surface of said objects.

281. An apparatus according to claim 280, and further comprising a transporting star arranged for transporting said objects from said first sterilizing device to said plurality of carousels.

282. An apparatus according to claim 279, wherein said sterilizing devices comprise a treating device, arranged for treating said objects with a sterilizing solution.

283. An apparatus according to claim 282, wherein said sterilizing devices further comprise a preheating device arranged for preheating said objects.

284. An apparatus according to claim 283, wherein said sterilizing devices further comprise a cooling device arranged for cooling said objects.

285. An apparatus according to claim 284, wherein said sterilizing devices further comprise a drying device arranged for drying said objects.

286. An apparatus according to claim 283, wherein said preheating device is positioned on a first carousel of said plurality of carousels.

287. An apparatus according to claim 286, wherein said treating device is positioned on said first carousel of said plurality of carousels in such a way as to interact with said objects after said preheating device.

288. An apparatus according to claim 286, wherein said sterilizing devices further comprise a cooling device arranged for cooling said objects, said cooling device being positioned on a second carousel of said plurality of carousels located downstream of said first carousel.

289. An apparatus according to claim 288, wherein said second carousel is provided with a regulating device that enables the duration of the cooling action of said cooling device on said objects to be varied.

290. An apparatus according to claim 288, and further comprising a first transporting star arranged for transporting said objects from said first carousel to said second carousels.

291. An apparatus according to claim 288, wherein said sterilizing devices further comprise a cooling device arranged for cooling said objects and a drying device arranged for drying said objects, said drying device being positioned on a third carousel of said plurality of carousels located downstream of said second carousels.

292. An apparatus according to claim 291, and further comprising a second transporting star arranged for transporting said objects from said second carousel to said third carousel.

293. An apparatus according to claim 286, wherein said treating device is positioned on a second carousel of said plurality of carousels located downstream of said first carousel.

294. An apparatus according to claim 293, and further comprising a first transporting star arranged for transporting said objects from said first carousel to said second carousel.

295. An apparatus according to claim 293, wherein said sterilizing devices further comprise a cooling device arranged for cooling said objects, said cooling means being positioned on a third carousel of said plurality of carousels located downstream of said second carousel.

296. An apparatus according to claim 295, wherein said third carousel is provided with a regulating device that enables the duration of the cooling action of said cooling device on said objects to be varied.

297. An apparatus according to claim 295, and further comprising a second transporting star arranged for transporting said objects from said second carousel to said third carousel.

298. An apparatus according to claim 295, wherein said sterilizing devices further comprise a drying device arranged for drying said objects, said drying device being positioned on a fourth carousel of said plurality of carousels located downstream of said third carousel.

299. An apparatus according to claim 298, and further comprising a third transporting star arranged for transporting said objects from said third carousel to said fourth carousel.

300. An apparatus according to claim 286, wherein said treating device is positioned on a second carousel of said plurality of carousels located downstream of said first carousel.

301. An apparatus according to claim 300, and further comprising a first transporting star arranged for transporting said objects from said first carousel to said second carousel.

302. An apparatus according to claim 300, wherein said sterilizing devices further comprise a cooling device arranged for cooling said objects, said cooling device being positioned on said second carousel of said plurality of carousels in such a way as to interact with said objects after said treating device.

303. An apparatus according to claim 302, wherein said sterilizing devices further comprise a drying device arranged for drying said objects, said drying device being positioned on a third carousel of said plurality of carousels located downstream of said second carousel.

304. An apparatus according to claim 303, and further comprising a second transporting star arranged for transporting said objects from said second carousel to said third carousel.

305. An apparatus according to claim 286, wherein said treating device is positioned on a second carousel of said plurality of carousels located downstream of said first carousel.

306. An apparatus according to claim 305, and further comprising a first transporting star arranged for transporting said objects from said first carousel to said second carousel.

307. An apparatus according to claim 305, wherein said sterilizing devices further comprise a cooling device arranged for cooling said objects, said cooling means being positioned on a third carousel of said plurality of carousel located downstream of said second carousel.

308. An apparatus according to claim 307, and further comprising a second transporting star arranged for transporting said objects from said second carousel to said third carousel.

309. An apparatus according to claim 307, wherein said sterilizing devices further comprise a drying device arranged for drying said objects, said drying device being positioned on said third carousel of said plurality of carousels in such a way as to interact with said objects after said cooling device.

310. An apparatus according to claim 282, wherein said treating device comprises a conveying device arranged for conveying into said objects a sterilizing solution for sterilizing said objects.

311. An apparatus according to claim 282, wherein said treating device comprises a heating arrangement arranged for heating a sterilizing solution for sterilizing said objects.

312. An apparatus according to claim 311, wherein said heating arrangement comprises a resistor arranged for absorbing energy from a source to supply it to said sterilizing solution.

313. An apparatus according to claim 312, wherein said heating arrangement further comprises a heat-accumulating device arranged for accumulating the heat released by said resistor and transferring it to said sterilizing solution.

314. A sterilizing apparatus for sterilizing objects, comprising a conduit for conveying a sterilizing solution to said objects, a resistor arranged for heating said solution flowing in said conduit, wherein said apparatus further comprises a heat-accumulating device arranged for accumulating the heat released by said resistor and transferring it to said solution.

315. A dosing apparatus for dosing a solution, in particular a sterilizing solution for sterilizing objects, comprising an accumulating chamber arrangement for accumulating said solution, a propelling device for propelling said solution along piping, wherein said apparatus further comprises pressure-reducing arrangement positioned downstream of said accumulating chamber arrangement and arranged for reducing the pressure of said sterilizing solution.

316. A method for treating objects comprising transporting along a curved path said objects by means of a plurality of rotating carousels comprising sterilizing said objects during said transporting with sterilizing devices arranged on said plurality of carousels, wherein said transporting comprises transferring said objects between a first carousel of said plurality of carousels and at least a further carousel of said plurality of carousels.

317. A method for sterilizing objects by means of a sterilizing solution, comprising conveying said solution to said objects, absorbing energy by means of a resistor, wherein there is provided for accumulating heat produced by said resistor and transferring it to said solution by means of a heat-accumulating device.

318. A dosing method for dosing a solution, in particular a sterilizing solution for sterilizing packaging units, comprising collecting said solution in an accumulating chamber arrangement, successively propelling said solution along piping, wherein there is provided for reducing the pressure of said solution by means of a pressure-reducing arrangement positioned downstream of said accumulating chamber arrangement.

319. An apparatus for treating objects comprising a rotating carousel arrangement for transporting said objects along a curved path, said rotating carousel arrangement comprising at least a treatment carousel for treating objects, and a sterilizing arrangement arranged on said carousel arrangement along said path, wherein said sterilizing arrangement comprises a sterilizing device that operates at ambient pressure.

320. A method for treating objects comprising transporting along a curved path said objects by means of a rotating carousel arrangement comprising at least a carousel, sterilizing said objects during said transporting with a sterilizing arrangement arranged on said carousel arrangement, wherein said sterilizing comprises sterilizing at ambient pressure.
